(19) United States
(12) Plant Patent Application Publication
(10) Pub. No.: US 2017/0118889 P1
(43) Pub. Date: Apr. 27, 2017

(54) Physocarpus plant named 'SMNPOTWG'

(50) ;

(71) Applicant: Timothy Wood, Spring Lake, MI (US)

(72) Inventor: Timothy Wood, Spring Lake, MI (US)

(21) Appl. No.: 14/756,865

(22) Filed: Oct. 22, 2015

(51) Int. Cl. A01H 5/00

(52) U.S. Cl. PLT/226

(57) ABSTRACT

A new and distinct Physocarpus cultivar named ‘SMNPOTWG’ is disclosed, characterized by a small plant form, early flowering, smaller leaves, mildew resistance and abundant red-purple colored flowers. The new variety is a Physocarpus, normally produced as an outdoor garden or container plant.

Latin name of the genus and species: Physocarpus opulifolius.

Variety denomination: ‘SMNPOTWG’.

### BACKGROUND OF THE INVENTION

The new Physocarpus cultivar is a product of a planned breeding program conducted by the inventor, Timothy D. Wood, in Grand Haven, Minn. The objective of the breeding program was to produce new Physocarpus varieties with unique foliage and powdery mildew resistance. The open pollenation resulting in this new variety was made during Summer of 2010.

The new Physocarpus ‘SMNPOTWG’ resulted from an open pollination in which seed was collected from an unnamed, unpatented proprietary seedling. Various Physocarpus were present in the field, making accurate identification of the pollen parent impossible. The new variety was identified as a potentially interesting selection in the Summer of 2013, at a commercial nursery in Grand Haven, Minn.

Asexual reproduction of the new cultivar ‘SMNPOTWG’ by softwood cuttings was first performed during the Summer of 2013, at commercial greenhouse in Grand Haven, Minn. Subsequent propagation has shown that the unique features of this cultivar are stable and reproduced true to type in 3 successive generations.

### SUMMARY OF THE INVENTION

The cultivar ‘SMNPOTWG’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘SMNPOTWG’ These characteristics in combination distinguish ‘SMNPOTWG’ as a new and distinct Physocarpus cultivar:
1. Smaller plant size.
2. Smaller leaves.
3. Yellow-Green leaves.
4. Good mildew resistance.
5. Flowering at a younger age.
6. Good performance in containers.

### PARENT COMPARISON

Plants of the new cultivar ‘SMNPOTWG’ are similar to plants of the parent, in most horticultural characteristics, however, plants of the new cultivar ‘SMNPOTWG’ differ in the following:
1. Improved resistance to mildew.
2. Flowers at a younger age than the parent variety.
3. ‘SMNPOTWG’ is a dwarf, the parent is full sized.
4. The new variety has Yellow-Green foliage, the parent has yellow foliage with red new growth.

### COMMERCIAL COMPARISON

Plants of the new cultivar ‘SMNPOTWG’ can also be compared to the commercial variety Physocarpus opulifolius ‘Bert Dart's Gold’, unpatented. The varieties are similar in most horticultural characteristics; however ‘SMNPOTWG’ differs in the following:
1. The new variety is a dwarf plant, this comparator is a semi-dwarf.
2. The new variety has smaller leaves than this comparator.
3. The new variety blooms more consistently and abundantly at a younger age than this comparator.

### BRIEF DESCRIPTION OF THE PHOTOGRAPHS

The accompanying photograph in FIG. 1 illustrates in full color a typical 2 year old plant of ‘SMNPOTWG’ grown in a 2 gallon container.

FIG. 2 illustrates in full color a typical inflorescence and foliage of ‘SMNPOTWG’ grown outdoors in Grand Haven, Minn., in a garden setting. The photographs were taken using conventional techniques and although colors may appear different from actual colors due to light reflectance it is as accurate as possible by conventional photographic techniques.

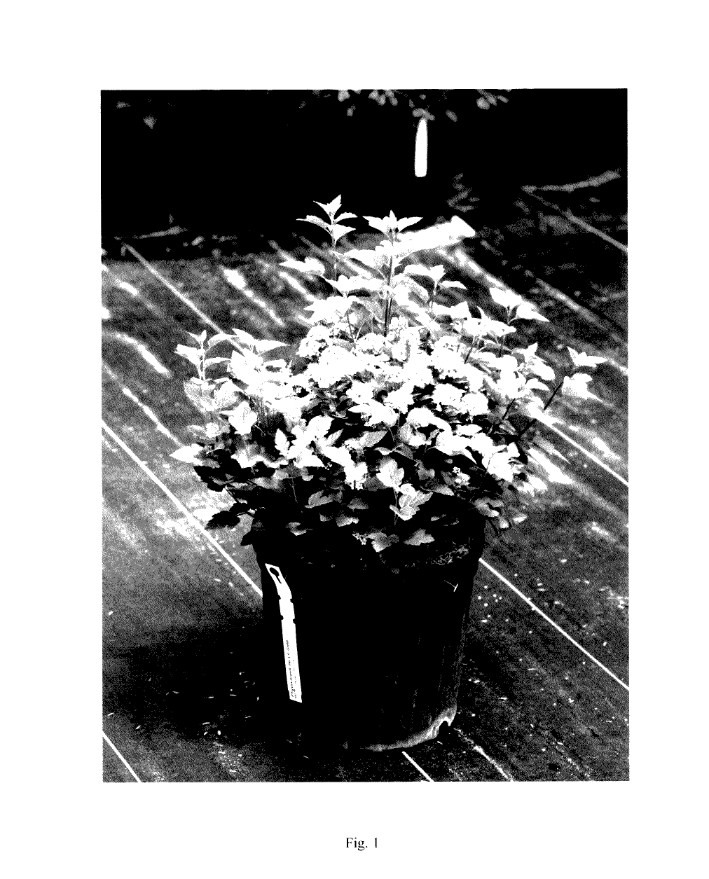

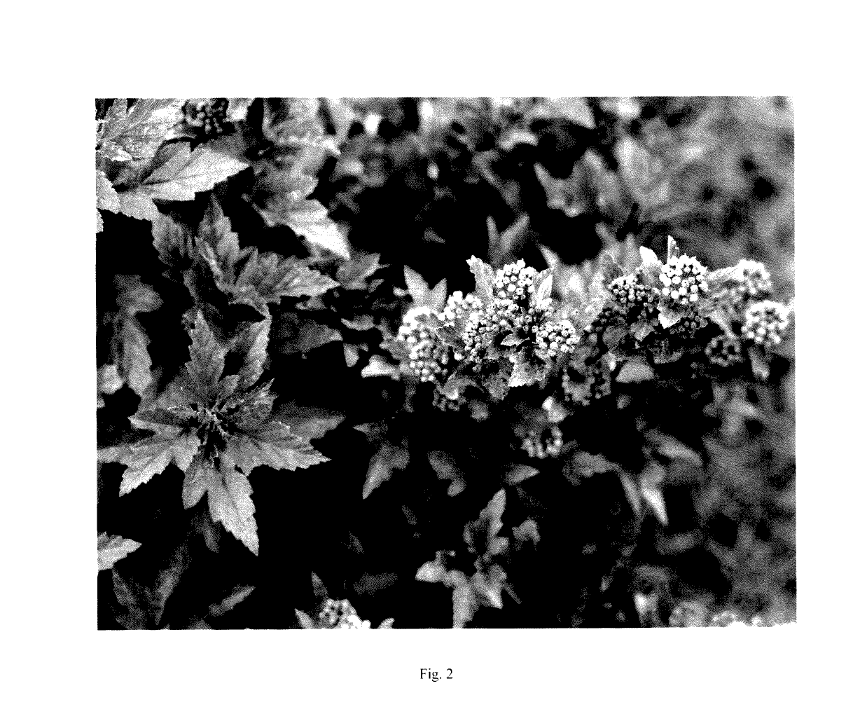

### DETAILED BOTANICAL DESCRIPTION

In the following description, color references are made to The Royal Horticultural Society Colour Chart 1996 except where general terms of ordinary dictionary significance are used. The following observations and measurements describe ‘SMNPOTWG’ plants grown outdoors in Grand Haven, Minn. This occurred during June of 2014. The plants were approximately 2 years old grown in 2 gallon containers. The growing temperature ranged from 18° C. to 27° C. during the day, and 5° to 10° C. at night. The field is un-shaded, giving bright, normal sunlight conditions. Measurements and numerical values represent averages of typical plant types.
Botanical classification: Physocarpus opulifolius ‘SMNPOTWG’.
Propagation:
   Time to initiate roots.—About 24 days at approximately 25° C.
   Root description.—Fine, and freely branching, both fibrous and fleshy.
   Time to produce a liner.—About 60 days at 24° C.
Plant:
   Plant type.—Shrub perennial.
   Age of plant described.—Approximately 2 years old.
   Growth habit.—Upright, rounded.
   Pot size of plant described.—2 gallon pot.
   Height.—Approximately 28 cm to top of foliage.
   Plant spread.—Approximately 40 cm.
   Growth rate.—Moderate.
   Plant vigor.—Strong.
   Branching characteristics.—Free, basal branching when pinched.
   Length of primary lateral branches.—Approximately 20-30 cm.
   Diameter of lateral branches.—Approximately 0.3 cm.
   Quantity of lateral branches.—Minimum average 50.
   Stem.—Appearance: Rounded, slight pentagonal shape Aspect/Angle: Some 20° others 90° Strength: Strong Stem pubescence: None Stem Color: Near RHS Yellow-Green 144B Bark color: Near RHS Brown 200D.
   Internode length.—Average range 2 to 3 cm.
Foliage:
   Leaf.—Arrangement: Alternate, single. Average Length: Approximately 4.5 cm. Average Width: Approximately 3.4 cm. Shape of blade: Cordate, Tri-lobed Apex: Acute. Base: Rounded Attachment: Stalked. Margin: Serrate-dentate. Texture of top surface: Smooth Texture of bottom surface: Smooth. Pubescence: None Color: Young foliage upper side: Near RHS Green 144C Young foliage under side: Near RHS Green 144C Mature foliage upper side: Near RHS Green 143C Mature foliage under side: Near RHS Green 139C Venation: Type: Palmate Venation color upper side: Near RHS Green 143C Venation color under side: Near RHS Green 139C. Petiole: Average Length: Approximately 1.7 cm. Diameter: Approximately 0.1 cm Petiole color upper side: Near RHS Green 143C Petiole color lower side: Near RHS Green 144B. Petiole Texture upper side; Glabrous, two ridges rim on the edges of the petiole. Petiole Texture lower side; Glabrous.
Flower:
   Natural flowering season.—May-June.
   Inflorescence type and habit.—Terminal and axillary rounded cyme.
   Quantity of flowers per inflorescence.—Average range 31 to 40.
   Inflorescence size.—Width: Approximately 3.5 cm. Height: Approximately 2.8 cm.
   Quantity of flowers per lateral stem.—200 to 800.
   Quantity of flowers per plant.—2000 to 8000.
   Individual flowers.—Flower form: single whorl of five petals Flower Aspect: Upright and outward Size: Diameter: Approximately 0.7 cm Length: Approximately 0.5 cm Depth: Approximately 0.4 cm Persistence: Persistent.
   Petal.—Petal arrangement: Whorl Number of petals per flower: 5 Petal appearance: flat, white, rounded Petal Shape: Obovate. Margin: Entire. Tip shape: Obovate Length: 0.3 cm Width: 0.2 cm Texture: Upper: Glabrous Lower: Glabrous.
   Petal color.—When opening: Upper surface: Outer edge near RHS Red-Purple 63C; rest near RHS White 155D Lower surface: Outer edge near RHS Red-Purple 63C; rest near RHS White 155D Fully opened: Upper surface: Near RHS White 155D Lower surface: Near RHS White 155D Fading: Upper surface: Near RHS White 155A Lower surface: Near RHS White 155A.
   Petalloids.—None present.
   Bud.—Shape: Globose Length: Approximately 0.3 cm Diameter: Approximately 0.3 cm Color: Near RHS White 155D, maturing to near RHS Red-Purple 62B.
   Sepals.—Shape: Triangular Arrangement: Whorl Length: Approximately 0.2 cm Width: Approximately 0.15 cm Margin: Entire Base: Connected, Obtuse Tip: Acuminate Texture: Pubescent Color: Immature: Near RHS Yellow-Green 144B Mature: Near RHS Yellow-Green 145C.
   Peduncle.—Length: Average 2.0 cm. Diameter: 0.2 cm Color: Near RHS Yellow-green 144B Orientation: 60° from stem, straight. Strength: Good Texture: Glabrous.
Reproductive organs:
   Stamens.—Number: 20 Filament length: Approximately 0.2 cm Filament color: Near RHS White 155A.
   Anthers.—Shape: Globose Length: Less than 0.1 cm Color: Immature: Near RHS Red-Purple 58D When Fading: Near RHS Greyed-Orange 177A Pollen: Some Pollen Color: Near RHS White 155A.
   Pistil.—Number: Average of 4 Length: Approximately 0.3 cm Style: Length: Approximately 0.3 cm Color: Near RHS White 155B Stigma: Shape: Club-shaped Color: Near RHS Yellow-green 153D Ovary color: Near RHS Yellow-Green 151D.
Other characteristics:
   Seeds and fruits.—Not observed to date.
   Garden performance.—Very good.
   Disease/pest resistance.—Good resistance to Powder Mildew. Neither resistance nor susceptibility to pests common to Physocarpus have been observed.
   Temperature tolerance.—The new variety tolerates temperatures between −31 to 38 ° C.

What is claimed is:

1. A new and distinct cultivar of Physocarpus plant named ‘SMNPOTWG’ as herein illustrated and described.
